(19) United States
(12) Patent Application Publication
(10) Pub. No.: US 2016/0100876 A1
(43) Pub. Date: Apr. 14, 2016

(54) SCREW HOLDER TO HOLD TINY SCREWS FOR APPLICATION ON BONE & CRANIUM DURING NEUROSURGERY

(71) Applicant: Ghansham Das Agarwal, Kolkata (IN)

(72) Inventor: Ghansham D. Agarwal, Kolkata (IN)

(21) Appl. No.: 14/372,427

(22) PCT Filed: Apr. 30, 2013
(86) PCT No.: PCT/IN13/00288

§ 371 Date: Jul. 15, 2014

(30) Foreign Application Priority Data
Feb. 7, 2013 (IN) 148/KOL/2013

(51) Int. Cl. A61B 17/88

(52) U.S. Cl. CPC A61B 17/8872

(57) ABSTRACT

This invention relates to a screw holder to hold tiny screws for application on bone & cranium during neurosurgery comprising of a handle integrally connected with an expandable tip for holding screw, wherein the tip comprising of a slit and a hole. It is associated with the following advantageous features:
Simple is construction
Efficient
Cost effective
Facilitates holding, accurate placement of screw and tightening.

A screw holder to hold tiny screws for application on bone & cranium during neurosurgery

### FIELD OF INVENTION

This invention relates to a screw holder to hold tiny screws for application on bone & cranium during neurosurgery. The screws are used for attaching plates & other implants with bone & cranium.

### BACKGROUND/PRIOR ART OF INVENTION

A head injury or operation on head is followed by fixing/repairing of the cranium using plates of biocompatible material such as titanium. These plates are very thin with thickness of 0.4-0.8 mm thick, which are fixed on cranium with screws. These screws are having very tiny diameter of 1.4-1.8 mm and length of 3-7 mm. Therefore, it is very difficult to hold these screws with gloves wet with blood. It is simply not feasible to hold and place these screws on bone accurately.

However, some designs were devised to overcome this problem. One such design provides a combination of slots on head of screw and tip of screw driver such that when tip is pressed into the slot on the head of screw, it locks with friction.

Another design provides a screw driver with dispenser kind of tube, wherein the screw is locked inside the screw driver.

The drawback with both these designs is that, screw is not held securely with driver head that slight force can dislodge the screw. During application and tightening screw, downward force is applied in which slight misdirection of force dislodges the screw from screw driver. Again, placement of screw locked with screw driver tip is not convenient/workable. Therefore, the surgeon is required to have a special skill to use such screws.

Hence, it is required to develop a screw holder to hold tiny screws for application on bone & cranium during neurosurgery, which can address the aforesaid drawbacks and function efficiently.

### OBJECTS OF INVENTION

An object of this invention is to propose a screw holder to hold tiny screws for application on bone & cranium during neurosurgery which overcomes disadvantage(s) associated with the prior art.

Another object of this invention is to propose a screw holder, which holds tiny screws securely during application.

Still another object of this invention is to propose a screw holder for tiny screws, which holds the screws till the screw is tightened & penetrated/attached with the cranium/bone.

Still another object of this invention is to propose a screw holder for tiny screws, use of which is very easy and does not require any special skill.

Still another object of this invention is to propose a screw holder for tiny screws, which facilitates placement of tiny screws at target point accurately.

Still another object of this invention is to propose a screw holder to hold tiny screws for application on bone & cranium during neurosurgery which is simple in construction.

Further object of this invention is to propose a screw holder to hold tiny screws for application on bone & cranium during neurosurgery which is efficient.

Further object of this invention is to propose a screw holder to hold tiny screws for application on bone & cranium during neurosurgery which is cost effective.

### STATEMENT OF INVENTION

According to this invention there is provided a screw holder to hold tiny screws for application on bone & cranium during neurosurgery comprising of a handle integrally connected with an expandable tip for holding screw, wherein the tip comprising of a slit and a hole.

### BRIEF DESCRIPTION OF THE ACCOMPANYING DRAWINGS

Further objects and advantages of this invention will be more apparent from the ensuing description when read in conjunction with the accompanying drawings and wherein:

FIG. 1 shows: first embodiment of screw holder of the present invention.

FIG. 2 shows: second embodiment of screw holder of the present invention.

FIG. 3 shows: third embodiment of screw holder of the present invention.

FIG. 4 shows: exploded view of the third embodiment.

FIGS. 5 and 6 shows: holding of screw in the screw holder and placement of the screw on the target point.

FIGS. 7 and 8 shows: tightening of the screw with screw driver.

FIG. 9 shows: bulging of lower portion of bush.

### DETAILED DESCRIPTION OF THE INVENTION WITH REFERENCE TO THE ACCOMPANYING DRAWINGS

The present invention discloses a novel screw holder to hold tiny screws, which are used during surgery for application on bone and cranium. The application of screws is made for attaching plates and other implants with bone and cranium.

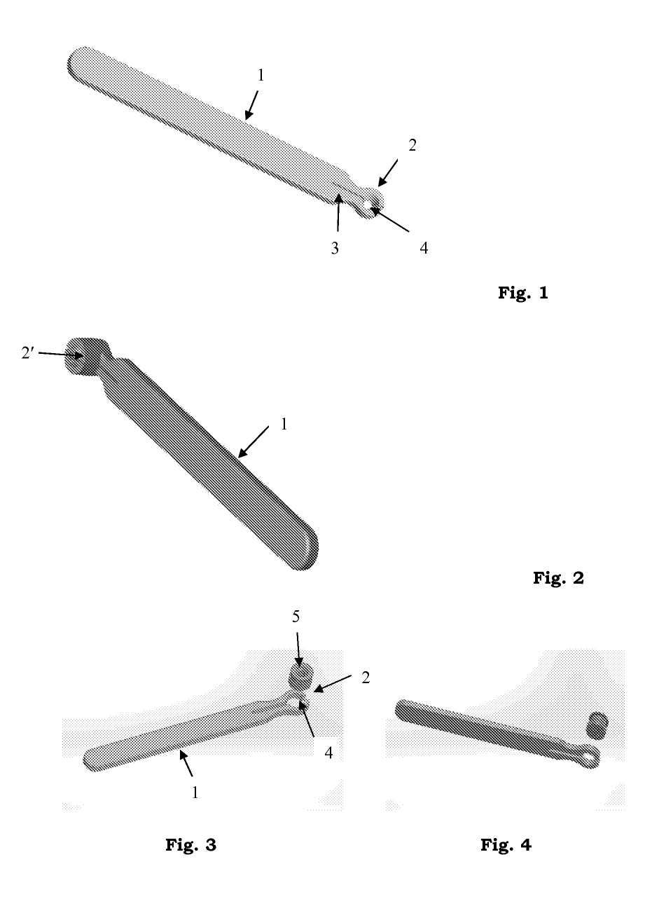

Reference may be made to FIG. 1, wherein the screw holder comprising a handle (1) and an expandable tip (2), which holds the screw. Said handle is integrally provided with the expandable tip. The tip is made expandable with a slit (3) and a hole (4) as shown in the FIG. 1. The slit is provided at one end of the screw holder passing through the hole of tip.

The outer profile of the tip can be of any shape preferably circular. However, the profile of the hole of the tip is according to the outer profile of the screw, which is preferably circular because the outer shape of the screw is generally circular. The holder of the instant invention is made of medical grade material which can be plastic with non toxic testing such as Polypropylene, Nylon and so many more without restricting scope of the invention to the same.

Further, there are several embodiments of the expandable tip as shown in FIGS. 2 and 3.

Referring to FIG. 2, wherein the expandable tip (2′) is thicker than the handle (1) to accommodate height of screw. This results in effective grip between the tip and screw, which facilitates application of screws for attaching plates or other implants with bone and cranium during surgery of head.

Now, referring to FIG. 3 indicating another embodiment of screw holder of the present invention. In this, a bush (5) is accommodated inside the hole (4) of the tip (2). The bush is made of soft resilient material such as rubber. This results in tight grip between the screw and bush.

The screw is held in the hole of screw holder tip /rubber bush till final tightening/ application of screw. As the screw is tightened, the holder tip/bush expands so as to accommodate the head of screw during its application for surgery on head.

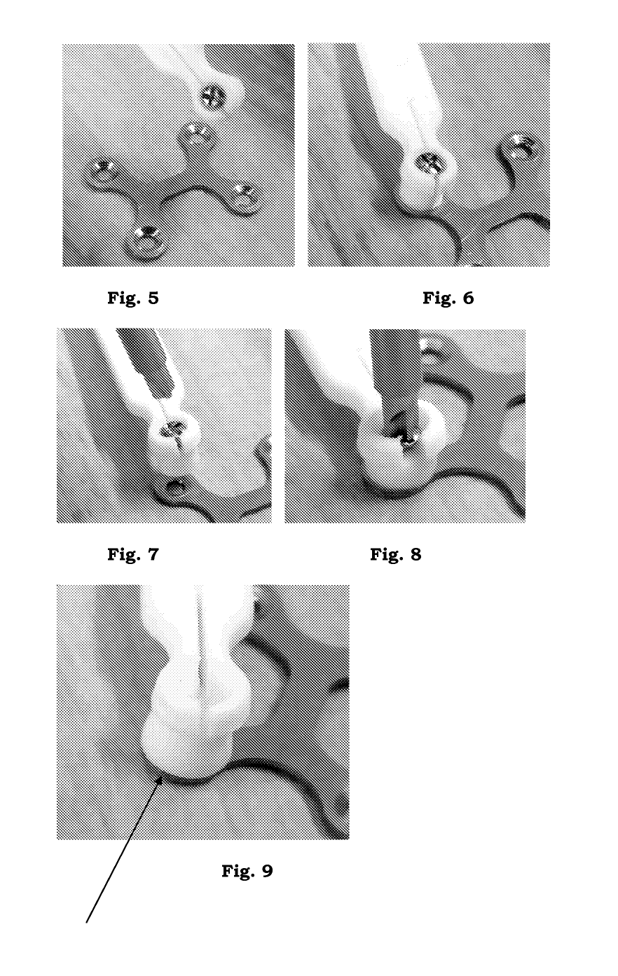

The same is indicated in FIG. 5-8, wherein the screw is held in the screw holder and placed on the target point very conveniently and easily. This is followed by tightening of the screw with the screw driver. The tip of holder undergoes expansion during tightening of the screw.

Further, reference may be made to FIG. 9, which shows bulging of lower portion of the bush after tightening of the screw. This is followed by removal of the screw holder and further tightening of screw to the required level.

It is now clearly evident from the aforesaid description that, holding, correct placement of screw and tightening is very convenient and easy with the help of screw holder of the invention.

### Advantageous Features

Simple is construction.
Efficient.
Cost effective.
Facilitates holding, accurate placement of screw and tightening

It is to be noted that the present invention is susceptible to modifications, adaptations and changes by those skilled in the art. Such variant embodiments employing the concepts and features of this invention are intended to be within the scope of the present invention, which is further set forth under the following claims:

1. A screw holder to hold tiny screws for application on bone and cranium during neurosurgery, comprising:
a handle integrally connected to a tip capable of expanding; and
wherein the tip comprises a slit and a hole.

2. The screw holder of claim 1, wherein the slit is provided at one end of the handle, and the slit passes through the hole.

3. The screw holder of claim 1, wherein the profile of the hole is provided according to an outer profile of a screw.

4. The screw holder of claim 1, wherein the outer profile of the tip is circular.

5. The screw holder of claim 1, the tip having a tip thickness;
the handle having a handle thickness; and
wherein the tip thickness is equal to the handle thickness.

6. The screw holder of claim 1, wherein the tip thickness is greater may be thicker than the handle thickness.

7. The screw holder of claim 1, wherein the tip further comprising a bush inside the hole of the tip.

8. The screw holder of claim 1, wherein the screw holder is made of a medical grade material.

9. (canceled)

10. The screw holder of claim 8, wherein the medical grade material is a plastic.

11. The screw holder of claim 8, wherein the medical grade material is selected from the group consisting of Polypropylene and Nylon.

12. The screw holder of claim 7, wherein the bush is made of rubber.
